(19) United States
(12) Patent Application Publication
(10) Pub. No.: US 2012/0271119 A1
(43) Pub. Date: Oct. 25, 2012

(54) AUTO-LOCKING SURGICAL RETRACTOR ASSEMBLY

(75) Inventor: William R. White, Parker, CO (US)

(73) Assignee: NSI-US, Inc., Peachtree City, GA (US)

(21) Appl. No.: 13/446,731

(22) Filed: Apr. 13, 2012

Related U.S. Application Data
(60) Provisional application No. 61/476,926, filed on Apr. 19, 2011.

(51) Int. Cl. A61B 1/32

(52) U.S. Cl. 600/228

(57) ABSTRACT

An auto-locking surgical retractor assembly has a retractor frame with a number of adjustable arms. Each adjustable arm has a U-shaped recess for removably engaging the post of a retractor blade. A spring-loaded locking pin protrudes slightly from the arm into the U-shaped recess to hold the post of retractor blade in place. The locking pin is temporarily retracted by insertion of the retractor blade post into the U-shaped recess, but subsequently extends to lock the retractor blade post in place. The user must exert sufficient force on the retractor blade to temporarily depress the spring-loaded locking pin in order to remove the retractor blade post from the U-shaped recess.

### RELATED APPLICATION

The present application is based on and claims priority to the Applicant's U.S. Provisional Patent Application 61/476,926, entitled “Auto-Locking Surgical Retractor Assembly,” filed on Apr. 19, 2011.

### BACKGROUND OF THE INVENTION

1. Field of the Invention

The present invention relates generally to the field of surgical retractors. More specifically, the present invention discloses an auto-locking surgical retractor assembly.

2. Statement of the Problem

Surgical retractors typically employ a retractor frame positioned outside and above the surgical site of the patient's anatomy to provide structural support for a number of removable retractor blades that retract the patient's tissue. One conventional type of retractor blade incorporates a round post extending upward from the top of the blade that can be removably engaged by a latch or locking mechanism on an adjustable arm extending from the retractor frame. A plurality of these retractor blades and adjustable arms may be required for proper retraction of tissue at a surgical site.

The locking mechanism ensures that the retractor blade remains fixed into the retractor frame, and thus prevents the risk of the retractor blade falling out during the surgical procedure. Conventional locking mechanisms add a degree of manual manipulation that the surgeon must be concerned with during the procedure. Other retractor frames do not include a locking mechanism. If there is no locking mechanism, or if the blade post is not aligned properly within the retractor, or if the surgeon inadvertently releases the locking mechanism during the procedure, the retractor blade can become dislodged inter-operatively. This can require repeated adjustment and manipulation time and cause frustration to the surgeon throughout the case. This can also lead to a possible tissue retraction failure and the potential for related negative clinical affects. At a minimum, it requires the surgeon to realign and reset the retractor frame and blades. This can lead to additional operating room time, additional operating room related costs, and the possibility of negative clinical outcomes. Therefore, a need exists for an auto-locking surgical retractor assembly that securely engages a retractor blade to the retractor frame, and also eliminates the need for the surgeon to manipulate a locking mechanism.

3. Solution to the Problem

The present retractor system incorporates an integrated self-locking mechanism that helps prevent the retractor blade from dislodging from the retractor frame. This self-locking design also allows the surgeon to attach the post of a retractor blade to the retractor frame easily with no manipulation by simply inserting the post of a retractor blade into a recess in an arm of the retractor frame, and it clicks in place.

Furthermore, this invention incorporates a self-aligning interface between the retractor frame and the retractor blade post, thereby eliminating the need for the surgeon to manually manipulate a mechanism to ensure the retractor blade locks in place. This system eliminates the need for the surgeon to constantly re-align the retractor blade within the retractor frame during the procedure. With the inclusion of the present mechanism, the retractor frame holds the blade securely without the need for constant adjustment, re-alignment, and the need for reinsertion of the retractor blade into the retractor frame during the procedure. This reduces operating room time, costs, and potentially improves clinical outcomes.

### SUMMARY OF THE INVENTION

This invention provides an auto-locking surgical retractor assembly having a retractor frame with a number of adjustable arms. Each adjustable arm has a U-shaped recess for removably engaging the post of a retractor blade. A spring-loaded locking pin protrudes slightly from the arm into the U-shaped recess to hold the retractor blade in place.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a perspective view of a portion of an assembled retractor frame 10 holding four retractor blades 20.

FIG. 2 is an exploded perspective view of a portion of a retractor frame assembly 10 engaging a retractor blade 20.

FIG. 3 is a detail perspective view of the end of an adjustable arm 12 of a retractor frame.

FIG. 4 is a top view of the distal segment of an adjustable arm 12.

FIG. 5 is a right side view of the distal segment of the adjustable arm 12 in FIG. 4.

FIG. 6 is a left side view of the distal segment of the adjustable arm 12 in FIG. 4

FIG. 7 is a front view of the distal segment of the adjustable arm 12 in FIG. 4.

FIG. 8 is a rear view of the distal segment of the adjustable arm 12 in FIG. 4.

FIG. 9 is a detail top view showing the post 22 of a retractor blade 20 being inserted into the recess 14 of the adjustable arm 12.

FIG. 10 is a vertical cross-sectional view corresponding to FIG. 9.

### DETAILED DESCRIPTION OF THE INVENTION

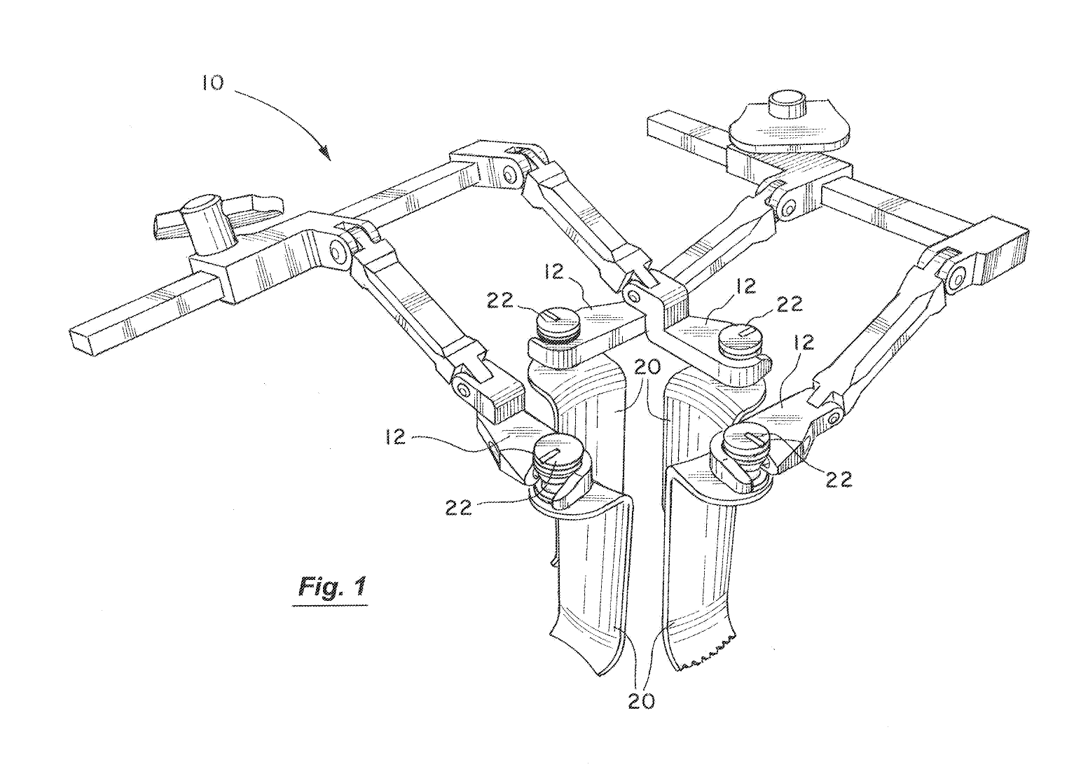

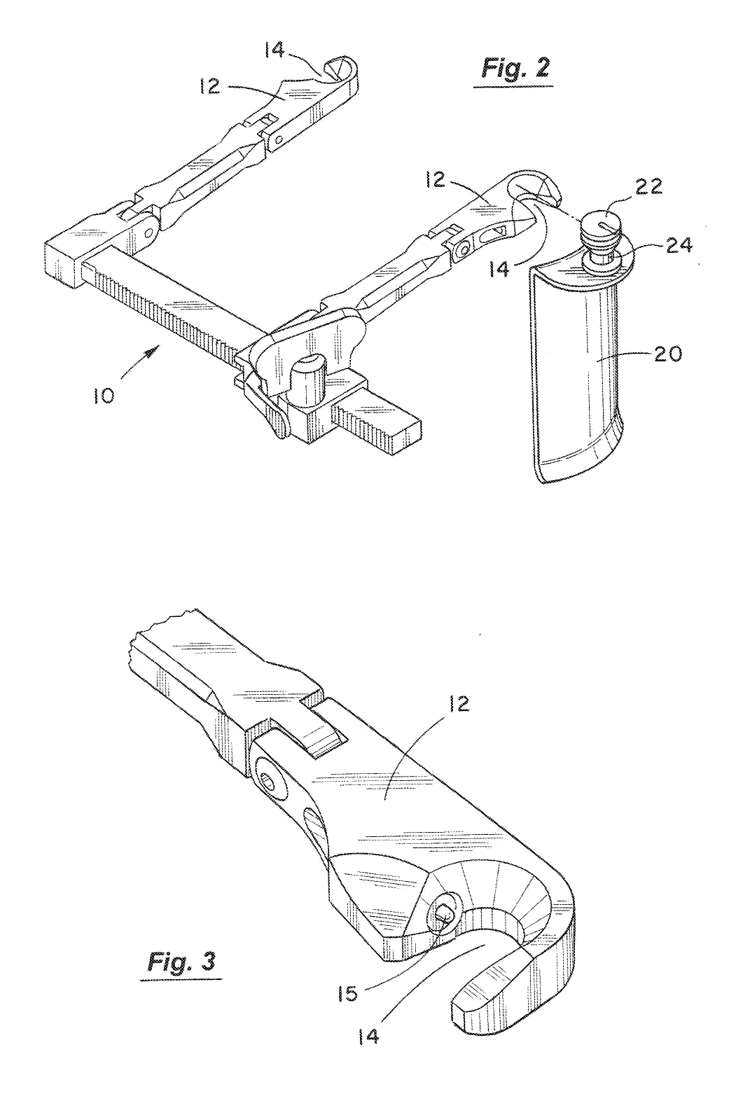

Turning to FIG. 1, a perspective view is shown of a portion of an assembled retractor frame 10 holding four retractor blades 20. FIG. 2 is an exploded perspective view of a portion of a retractor frame assembly 10 engaging a retractor blade 20. When used in an operating room, the retractor frame is typically positioned outside and above the surgical site of the patient's anatomy to provide structural support for retractor blades that retract the patient's tissue. The retractor frame 10 is equipped with a number of adjustable arms 12, each of which can be moved into a desired position relative to the surgical site for engaging a retractor blade 20. Each adjustable arm 12 can include a base at its proximal end with a ratchet and locking mechanism that allows the arm to slide along one of the bars of the retractor frame. The adjustable arm 12 can also be equipped with a number of joints or hinge mechanisms that allow the arm 12 to flex and pivot.

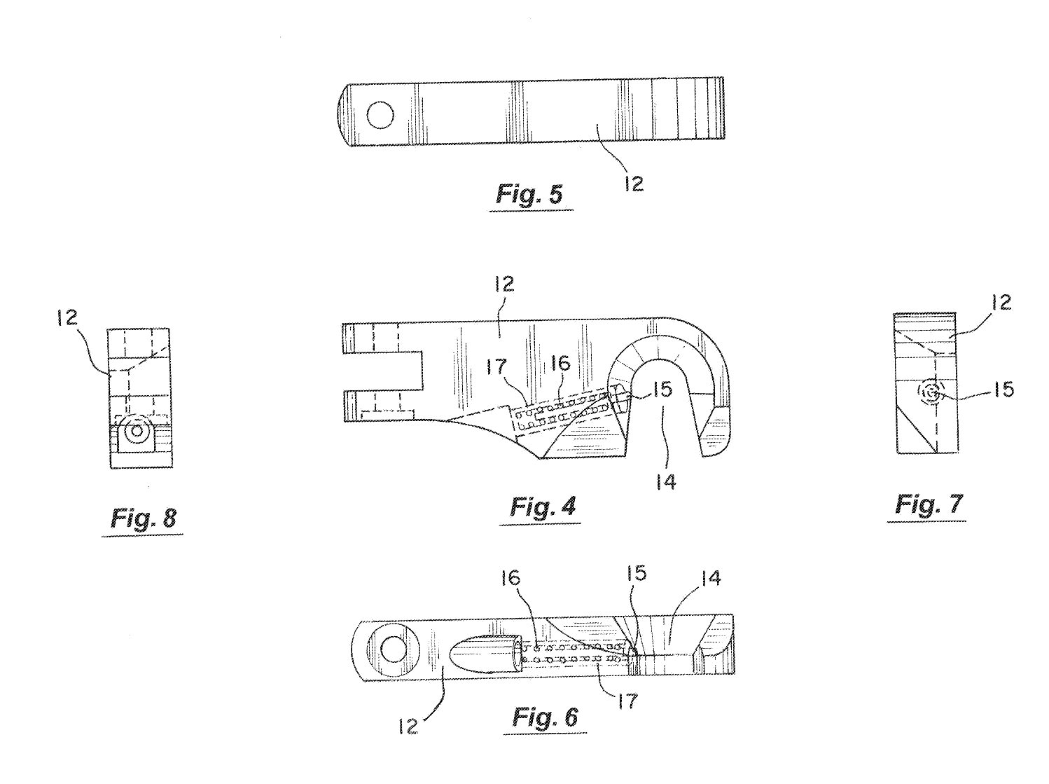

As depicted in FIGS. 3 and 4, the distal end of the adjustable arm 12 includes a generally U-shaped recess 14 for receiving and holding a retractor blade 20. The U-shaped recess 14 extends in a plane from a curved or rounded base, with opposing sides or edges defining a channel.

As illustrated in FIG. 2, the retractor blade 20 incorporates a round post 22 extending upward from the top of the blade for sliding, removable engagement in the plane of the U-shaped recess 14 on an adjustable arm 12. Preferably, the blade post 22 has a neck 24 with a substantially circular cross-section and a reduced diameter for sliding engagement with the walls of the U-shaped recess 14 on the adjustable arm 12. This U-shaped recess 14 should have dimensions and a cross-sectional shape that are complementary to those of the neck 24 of the blade post 22. In the specific embodiment shown in the drawings, the upper portion of the neck 24 has a tapered conical cross-section leading to a lower cylindrical section having a reduced radius. The walls of the U-shaped recess 14 have corresponding tapered conical contours to slide against the conical surfaces of the neck 24 of the blade post 22, while the smaller cylindrical section of the neck 24 slides between opposing lower vertical walls in the U-shaped recess 14. These conical and tapered surfaces help to guide and align the blade post 22 into proper three-dimensional position and alignment for sliding engagement into the U-shaped recess 14 in the adjustable arm 12.

The present invention also includes a spring-loaded locking pin 15 that protrudes slightly into the U-shaped recess 14 from the adjustable arm 12, as shown in FIG. 3. FIG. 4 is a top view of the distal segment of an adjustable arm 12 showing this locking pin 15. Corresponding right side, left side, front and rear views of this distal segment of the arm 12 are shown in FIGS. 5-8, respectively. The body of the pin 15 is held in a cylindrical chamber 17 within the body of the adjustable arm 12, as illustrated in these figures. A spring 16 biases the pin 15 forward in the chamber 17, so that the head of the pin 15 protrudes slightly into the channel of the U-shaped recess 14.

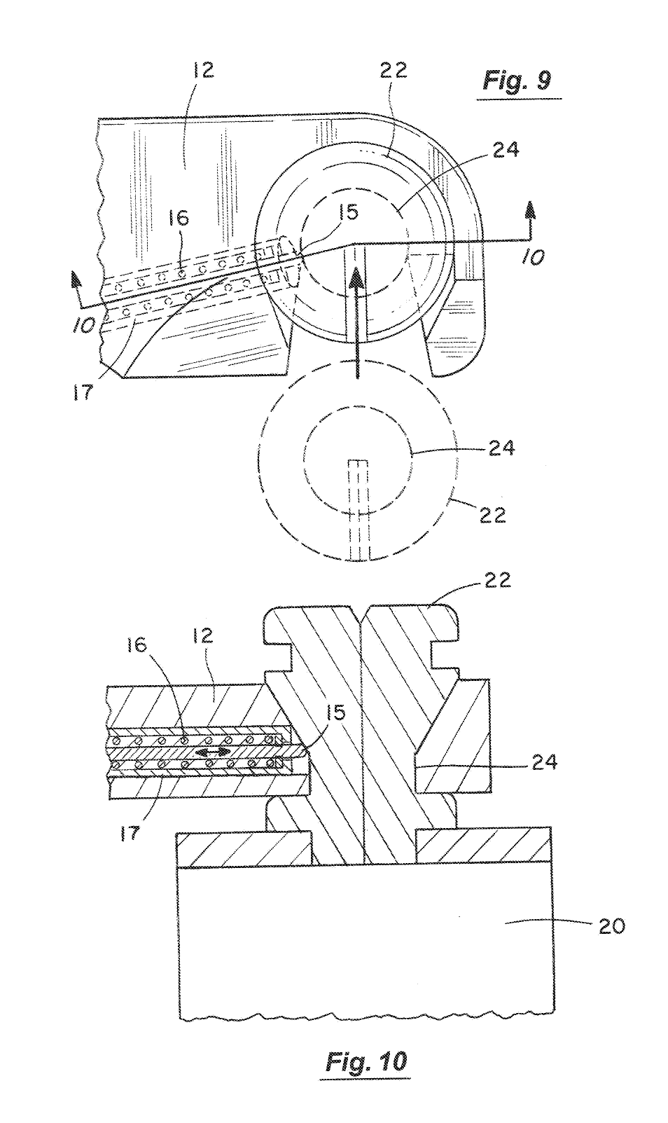

As the post 22 of a retractor blade 20 is inserted into the U-shaped recess 14, the locking pin 15 is momentarily pushed back or deflected into a more retracted position by contact with the neck 24 of the retractor blade post 22, which slightly compresses the biasing spring 16. When the blade post 22 is completely seated in the recess 14 in the adjustable arm 12, the pin 15 is allowed to protrude slightly more and contacts the neck 24 of the retractor blade post 22 at a point slightly outward from, or beyond the central axis of the retractor blade post 22, as shown in FIG. 9. FIG. 10 is a vertical cross-sectional view corresponding to FIG. 9.

Preferably, the locking pin 15 protrudes into the U-shaped recess 14 along an axis that is inclined (e.g., by a small angle) toward the curved base of the U-shaped recess 14 as shown in FIGS. 4 and 9. In addition, the locking pin 15 is located on the edge the U-shaped recess at a distance from the curved base of the U-shaped recess 14 that is greater than the radius of the neck 24, as illustrated in FIG. 9. In other words, the tip of the locking pin 15 contacts the retractor blade post 22 at a point beyond the region of maximum width of the neck 24 of the retractor blade post 22, so that any outward movement by the retractor blade post 22 to unseat itself from the U-shaped recess 14 in the adjustable arm 12 causes the retractor blade post 22 to push against the head of the locking pin 15 and compress the spring 16, thereby tending to hold the retractor blade post 22 in place. This prevents movement of the retractor blade post 22 in the U-shaped recess 14, and also retains the retractor blade post 22 in the U-shaped recess 14 unless sufficient outward force is exerted by the healthcare provider to overcome the biasing force exerted by the spring 16 to allow the locking pin 15 to retract.

Preferably, the post 22 of a retractor blade 20 slides into engagement with the adjustable arm 12 with minimal effort. In addition, the locking pin 15 and the shape of the recess 14 can be designed to create an audible or tactile “click” or “pop” to confirm that the retractor blade 20 has successfully engaged the adjustable arm 12. The angle of the locking pin 15 relative to the neck 24 of the blade post 22 and the shape of the head of the pin 15 can also be designed so that a greater force is required to detach a retractor blade 20 from the adjustable arm 12, than is required to initially slide the blade post 22 into engagement with the adjustable arm 12.

The embodiment of the present invention shown in the drawings employs an elongated locking pin 15. It should be understood that other shapes and configurations could be substituted. For example, a spring-loaded ball or sliding member that is biased to protrude into the U-shaped recess 14 of the adjustable arm 12 could be employed.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

I claim:

1. An auto-locking surgical retractor assembly comprising:
a retractor blade having a post with a neck having a reduced diameter;
a retractor frame;
an adjustable arm having a proximal end attached to the retractor frame and a distal end with a U-shaped recess in a plane for receiving the neck of the retractor blade post in sliding engagement in said plane; and
a spring-loaded locking pin biased to protrude from the adjustable arm into the U-shaped recess; said spring-loaded locking pin being temporarily retracted by insertion of the retractor blade post into the U-shaped recess, and subsequently extending to lock the retractor blade post in the U-shaped recess until sufficient force is exerted on the retractor blade to temporarily depress the spring-loaded locking pin and thereby allow removal of the retractor blade post from the U-shaped recess.

2. The auto-locking surgical retractor assembly of claim 1 wherein the neck of the retractor blade post comprises a tapered section adjacent to the cylindrical section.

3. The auto-locking surgical retractor assembly of claim 1 wherein the spring-loaded pin further comprises a pin housed within a cylindrical chamber in the adjustable arm having a head protruding into the U-shaped recess, and a spring compressed within the cylindrical chamber to exert a biasing force on the pin.

4. The auto-locking surgical retractor assembly of claim 1 wherein the neck of the retractor blade post has a conically-tapered collar leading to a cylindrical section, and wherein the distal end of the adjustable arm has complementary conical contours adjacent to the U-shaped recess to align the neck of the retractor blade post for sliding engagement into the U-shaped recess.

5. The auto-locking surgical retractor assembly of claim 1 wherein the neck of the retractor blade has a region of maximum width on insertion into the U-shaped recess, and wherein the spring-loaded locking pin protrudes into the U-shaped recess at a point beyond the region of maximum width of the neck.

6. The auto-locking surgical retractor assembly of claim 1 wherein the U-shaped recess in the distal end of the adjustable arm has a curved base between opposing sides, and wherein the spring-loaded locking pin protrudes into the U-shaped recess along an axis inclined toward the curved base of the U-shaped recess.

7. The auto-locking surgical retractor assembly of claim 1 wherein the U-shaped recess in the distal end of the adjustable arm has a curved base between opposing sides, and wherein neck of the retractor blade has a substantially circular cross-section with a radius, and wherein the spring-loaded locking pin protrudes into the U-shaped recess at a distance from the curved base greater than the radius of the neck.

8. An auto-locking surgical retractor assembly comprising:
a retractor blade having a post with a circular neck having a reduced radius;
a retractor frame;
an adjustable arm having a proximal end attached to the retractor frame and a distal end with a U-shaped recess having a curved base between opposing sides in a plane; said U-shaped recess receiving the neck of the retractor blade post in sliding engagement in said plane; and
a spring-loaded locking pin biased to protrude from the adjustable arm into the U-shaped recess along an axis inclined toward the curved base of the U-shaped recess at a distance from the curved base greater than the radius of the neck; said spring-loaded locking pin being temporarily retracted by insertion of the retractor blade post into the U-shaped recess, and subsequently extending to lock the retractor blade post in the U-shaped recess until sufficient force is exerted on the retractor blade to temporarily depress the spring-loaded locking pin and thereby allow removal of the retractor blade post from the U-shaped recess.

9. The auto-locking surgical retractor assembly of claim 8 wherein the neck of the retractor blade post further comprises a tapered section adjacent to a cylindrical section.

10. The auto-locking surgical retractor assembly of claim 8 wherein the spring-loaded pin further comprises a pin housed within a cylindrical chamber in the adjustable arm having a head protruding into the U-shaped recess, and a spring compressed within the cylindrical chamber to exert a biasing force on the pin.

11. The auto-locking surgical retractor assembly of claim 8 wherein the neck of the retractor blade post has a conically-tapered collar leading to cylindrical section, and wherein the distal end of the adjustable arm has complementary conical contours adjacent to the U-shaped recess to align the neck of the retractor blade post for sliding engagement into the U-shaped recess.
